(19) United States
(12) Patent Application Publication
(10) Pub. No.: US 2019/0262256 A1
(43) Pub. Date: Aug. 29, 2019

(54) COSMETIC OR PHARMACEUTICAL COMPOSITION FOR SKIN TAUTENING, SKIN FIXATION AND WRINKLE REDUCTION

(71) Applicant: Mayser Pharma AG, Stans (CH)

(72) Inventor: Klaus-Uwe BUSCH, Luzern, Schweiz (CH)

(73) Assignee: Mayser Pharma AG, Stans (CH)

(21) Appl. No.: 16/333,844

(22) PCT Filed: Sep. 20, 2017
(86) PCT No.: PCT/EP2017/073717

§ 371 Date: Mar. 15, 2019

(30) Foreign Application Priority Data
Sep. 20, 2016 (DE) 10 2016 117 728.8

(51) Int. Cl. A61K 8/86; A61K 8/58; A61Q 9/04; A61Q 19/00

(52) U.S. Cl. CPC A61K 8/86; A61Q 19/00; A61Q 9/04; A61K 8/585

(57) ABSTRACT

A cosmetic or pharmaceutical composition contains at least one polymer precursor and at least one polymerization catalyst in the presence of which the at least one polymer precursor polymerizes under the action of light. Also, a process for skin tautening, skin fixation and/or wrinkle reduction includes an elastic, wearable polymer layer being formed on the skin when a polymer precursor and a polymerization catalyst are topically applied on the skin area to be treated and the skin area to be treated is subsequently exposed to light.

### SUBJECT OF THE INVENTION

The present invention relates to a cosmetic or pharmaceutical composition for skin tautening, skin fixation and wrinkle reduction as well as a process for cosmetic or pharmaceutical skin tautening, skin fixation and wrinkle reduction using the composition according to the invention.

### BACKGROUND OF THE INVENTION

The skin is an organ with different physiological functions. In particular it serves to protect against external influences, such as e.g. extreme temperatures, radiation or microorganisms. Over the years these external factors lead to ageing of the skin, which is compounded by further factors such as stress, diseases or chemical reagents in personal and beauty care products. The process of skin ageing becomes increasingly visible from the age of 30 and leads to the formation of wrinkles and lines and a loss of elasticity and tonicity of the skin.

Besides the ageing due to external factors, there is also intrinsic skin ageing due to the genetically controlled reduced reactivity of skin cells. This leads to a depletion of elastin and collagen in the tissue. In addition the water and fat content of the skin reduces significantly, which leads to the complexion appearing relatively dry and the increased formation of wrinkles and cracks in the skin.

### STATE OF THE ART

In order to counteract the process of skin ageing and/or in order to conceal the outwardly apparent effects of skin ageing, various cosmetic or pharmaceutical compositions have been developed.

For example, DE 103 08 852 A1 proposes, as agent for the reduction of wrinkle formation in the skin, an aqueous composition which contains spasmolytics embedded in liposomes or cerasomes.

DE 10 2009 001 710 A1 describes a cosmetic composition for the reduction of skin wrinkles, which contains an active-ingredient combination comprising hyaluronic acid and a β-glucan in an active-ingredient carrier comprising sphingolipids and/or galactolipids.

EP 2 259 765 B1 discloses a cosmetic method for smoothing wrinkles and fine lines using an interpolymer. This composition contains at least one polyurethane and at least one copolymer of vinylpyrrolidone and a silicone monomer.

EP 2 382 961 A2 relates to a cosmetic composition for optical concealing of wrinkles by means of silicone elastomers in oil-in-water emulsions.

EP 1 299 080 A2 describes skin care compositions containing silicone elastomers. These ensure the therapeutic regulation of visible and/or fillable unevenness of mammalian skin including unevenness of the skin texture and of the skin colour.

Langer et al. Nature Materials, 15, 911-918 (2016) discloses the use of elastic, wearable crosslinked polymer layers on the skin, which can mimic the properties of normal, youthful skin. The crosslinked polysiloxane-based materials can be adjusted in a targeted manner in terms of their elasticity and tensile strength. A platinum compound (Karstedt's catalyst) is used as polymerization catalyst, which is applied in a second application step in order to initiate the polymerization and crosslinking.

The effect of the various preparations known from the state of the art is often not as satisfactory as expected and is also often not suitable for permanently reducing the depth and extent of skin wrinkles that are already present. Conventional cosmetic compositions which contain silicone compounds are in addition very laborious to apply and moreover problematic in the case of long-term application.

### OBJECT

Accordingly, there is a need for a cosmetic or pharmaceutical composition which is more suitable for reducing the depth and extent of skin wrinkles than the preparations known from the state of the art, and the object of the present invention was therefore to provide a correspondingly improved composition.

### DESCRIPTION OF THE INVENTION

This object is achieved according to the invention by a cosmetic or pharmaceutical composition of the type described at the beginning, which is suitable for topical application to the skin and is additionally characterized in that it contains at least one polymer precursor and at least one polymerization catalyst, in the presence of which the at least one polymer precursor polymerizes under the action of light.

After topical application the composition according to the invention is suitable for producing on the skin a polymer layer with an extremely pleasant sensory impression within a few minutes, preferably within a few seconds. This polymer layer is among other things outstandingly suitable for tautening the skin or for concealing wrinkles and/or small lines in the treated skin area.

The polymer layer obtained according to the invention is wearable on the skin, i.e. is tolerated without a negative skin reaction even over several hours (preferably at least 6 hours, at least 12 or at least 24 hours) and days (up to 1 day, up to 3 days or even up to 7 days) of use on the skin. The polymer layer obtained according to the invention is preferably elastic for this purpose.

The term “cosmetic composition” is to be understood here as meaning a combination of chemical substances that is suitable for cosmetic use. The term “pharmaceutical composition” is to be understood here as meaning a combination of chemical substances that is suitable for pharmaceutical use.

Cosmetic compositions used serve merely for personal and beauty care, or for obtaining, restoring or improving the beauty of the human body, in the present case, in particular, of the skin. In comparison, pharmaceutical compositions serve primarily to prevent or heal disease states or at least to eliminate or alleviate the symptoms thereof. Pharmaceutical compositions contain corresponding active ingredients and come under pharmaceutical law—in contrast to purely cosmetic compositions.

The composition according to the invention is intended for topical application to the skin and is accordingly also suitable for this purpose. Within the meaning of the present invention, by “topical application to the skin” is meant the application of the cosmetic or pharmaceutical composition to a skin area to be treated.

The term “skin” refers to the skin of living beings, in particular that of humans, and in the context of the present invention encompasses both the skin layers of the epidermis, dermis and subcutis, as a whole or in each case taken individually, and the skin appendages, such as e.g. the hair, nails, sebaceous and sweat glands, as a whole or in each case taken individually. In preferred embodiments of the invention the term refers only to the outer skin and/or outer skin appendages.

The cosmetic or pharmaceutical composition according to the invention contains at least one polymer precursor which polymerizes in the presence of a polymerization catalyst under the action of light.

Within the meaning of the present invention, a “polymer precursor” is a number of at least one monomer, which contains at least two groups reactive in the catalyzed polymerization reaction and from which a polymer forms in the polymerization reaction.

Within the meaning of the present invention, a “polymerization catalyst” is a chemical substance, in the presence of which the polymerization reaction proceeds more quickly and/or at lower temperatures and/or under the action of less energy radiation than would be the case in the absence of the polymerization catalyst. Due to the presence of the polymerization catalyst a polymer can form within a few minutes or preferably within a few seconds under temperature conditions that are suitable for cosmetic use on the skin.

The term “polymerization” is to be understood here as denoting the process in which a polymer is formed. Within the meaning of the present invention, a polymer is formed when there is a degree of polymerization of at least 5, preferably at least 10, still more preferably at least 20.

The number of monomer units in a polymer molecule is referred to as the degree of polymerization. It is identical to the quotient of the average molar mass of the polymer and of the molar mass of its repeat unit.

In a preferred embodiment the polymer formed is a crosslinked polymer, thus a polymer in which the polymer molecules are at least partially linked to a three-dimensional network.

The polymerization reaction is effected according to the invention in the presence of a polymerization catalyst and under the simultaneous action of light.

The phrase “in the presence of which” is understood here such that polymer precursor and polymerization catalyst in the cosmetic or pharmaceutical composition have to be present in one and the same mixture on the skin, in any case after the topical application has been effected. This can be achieved either in that polymer precursor and polymerization catalyst are applied in a ready-made mixture or sequentially one after the other with a time gap.

The phrase “under the action of light” is understood here such that the polymerization reaction catalyzed by the polymerization catalyst either does not proceed at all or in any case not in the desired manner or not at the required speed without the action of light. For the formation of the polymer the cosmetic or pharmaceutical composition therefore has to be exposed to light after the topical application thereof, wherein this light can come from an artificial radiation source or from a natural radiation source (e.g. sunlight).

By the term “light” is meant here radiation in the wavelength range of from 200 nm to 800 nm, preferably of from 380 nm to 800 nm, particularly preferably 550 nm to 780 nm. Radiation in the range of from 200 nm to 400 nm ensures a faster formation of the polymer layer, whereas radiation in the range of from 380 nm to 800 nm is associated with a polymerization reaction which is slightly slower, but in return is slightly more gentle for the patient.

The expression of time for the polymerization, “within a few minutes”, preferably encompasses a period of less than 15 minutes, still more preferably less than 10 minutes. In particular embodiments, the polymerization is effected within from 1 to 10 minutes, still more preferably within from 1 to 5 minutes. The expression of time for the polymerization, “within a few seconds”, preferably encompasses a period of less than 60 seconds, still more preferably less than 30 seconds. In particular embodiments, the polymerization is effected within from 5 to 60 seconds, still more preferably within from 5 to 30 seconds.

In a cosmetic or pharmaceutical composition according to the invention, the polymer precursor can be a siloxane. In a preferred embodiment, the siloxane is a polyether-modified siloxane. The advantage of polyether-modified siloxanes is an improvement in the flow and the surface smoothness of the composition.

A preferred embodiment of the cosmetic or pharmaceutical composition according to the invention contains, as polymer precursor, either
a) a combination of
   i) at least one polyorganohydrosiloxane with reactive silane groups for the hydrosilylation, with the following structure:

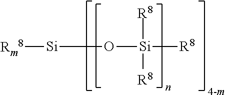

wherein R⁸ represent independent radicals selected from a group consisting of alkyl groups, cycloalkyl groups, epoxide groups, isocyanate groups, amino groups, alcohol groups, ether groups, ester groups, phenyl groups and hydrogen, provided that at least two, but not more than half of all R⁸ groups in the siloxane are hydrogens, and wherein m is 0, 1, 2 or 3 and n is a number with a value between 1 and 3000,
   and
   ii) at least one polyorganosiloxane, which contains unsaturated groups for the hydrosilylation, with the following structure:

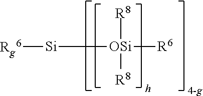

wherein R⁶ represent independent radicals selected from a group consisting of non-halogenated or halogenated ethylenically unsaturated groups, non-halogenated or halogenated alkyl groups, non-halogenated or halogenated cycloalkyl groups, epoxide groups, isocyanate groups, amino groups, alcohol groups, ether groups, ester groups, phenyl groups, provided that at least 70% of all R⁶ groups contain vinyl groups or other alkenyl groups and wherein h is a number with a value between 1 and 3000 and g is 0, 1, 2, or 3,
or
b) at least one polyorganohydrosiloxane with the following structure, which contains unsaturated functions and also silane groups for the hydrosilylation in one molecule:

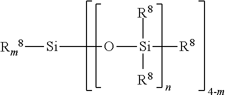

wherein R⁸ represent independent radicals selected from a group consisting of alkyl groups, cycloalkyl groups, phenyl groups, epoxide groups, isocyanate groups, amino groups, alcohol groups, ether groups, ester groups, hydrogen and non-halogenated or halogenated ethylenically unsaturated groups, provided that at least two, but not more than half of all R⁸ groups in the siloxane are hydrogens and furthermore provided that at least two of the R⁸ groups contain vinyl groups or alkenyl groups, wherein m is 0, 1, 2 or 3 and n is a number with a value between 1 and 3000.

A preferred embodiment of the cosmetic or pharmaceutical composition according to the invention in accordance with the above-named alternative a) contains, as polymer precursor, a combination of i) and ii), wherein at least 70% of all R⁶ groups are vinyl groups or other alkenyl groups.

A preferred embodiment of the cosmetic or pharmaceutical composition according to the invention in accordance with the above-named alternative b) contains, as polymer precursor, a polyorganohydrosiloxane, wherein at least two of the R⁸ groups are vinyl groups or alkenyl groups.

The composition according to the invention contains a polymerization catalyst, thus a chemical substance, in the presence of which the polymerization proceeds more quickly and/or at lower temperatures and/or under the action of less energy radiation than would be the case in the absence of the polymerization catalyst.

In particular embodiments of the invention, the polymerization catalyst is a compound which comes under the general formula (II),

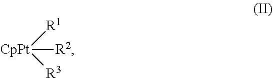

wherein Cp represents a cyclopentadienyl group which is q-bonded to the platinum atom, wherein the cyclopentadienyl group is optionally mono- or polysubstituted by radicals which do not interfere with the hydrosilylation reaction, and each of the radicals R¹, R² and R³ represents an aliphatic or aromatic group with 1-18 carbon atoms, wherein R¹, R² and R³ are σ-bonded to the platinum atom, and wherein the composition additionally contains at least one photosensitizer which is suitable for transferring the absorbed light energy to the polymerization catalyst.

In particular embodiments of the invention, in addition to the polymerization catalyst a photosensitizer has to be provided which absorbs light in the range used here and can provide the thus-absorbed energy in a suitable form for the purpose of catalyzing the polymerization reaction.

Photosensitizers which are suitable for the present invention are those compounds which can absorb light within the range used according to the invention and can transfer energy to the platinum complex. Corresponding photosensitizers are preferably selected from polycyclic aromatic compounds, preferably aromatic compounds with 2 to 5 rings, thioxanthones, 2-chlorothioxanthones, 2-isopropylthioxanthones and aromatic compounds with at least one ketone chromophore.

The compounds of the photosensitizer can be substituted by any desired substituent which does not impair the ability of the compound of the photosensitizer or of the hydrosilylation catalyst to absorb light and transfer energy. Examples of typical substituents are: alkyl, alkoxy, aryl, aryloxy, aralkyl, alkaryl and halogen substituents.

Anthracene, 9-vinylanthracene, 9,10-dimethylanthracene, 9,10-dichloroanthracene, 9,10-dibromoanthracene, 9,10-diethylanthracene, 9,10-diethoxyanthracene, 2-ethyl-9,10-dimethylanthracene, naphthacene, pentacene, benz[a]arithracene, 7,12-dimethylbenz[a]anthracene and azulene can be named as specific examples of polycyclic aromatic photosensitizers which are suitable for the invention.

2-Chlorothioxanthone, 2-isopropylthioxanthone, thioxanthone, anthraquinone, benzophenone, 1-chloroanthraquinone and bianthrone can be named as specific examples of aromatic ketones which are suitable as photosensitizer for the present invention.

In a particular embodiment of the above-described variant of the present invention in which a photosensitizer is used, a platinum complex, with an η-bonded cyclopentadienyl group in combination with a photosensitizer, is utilized for the catalysis of the polymerization. The hydrosilylation process is induced by visible light, wherein the photosensitizer absorbs the visible light and transfers the energy to the platinum complex.

In particular embodiments of the invention, an additional photosensitizer need not be provided, namely when the polymerization catalyst can itself absorb light in the range used here and can provide the thus-absorbed energy in a suitable form for the purpose of catalyzing the polymerization reaction.

An example of a polymerization catalyst suitable for the present invention, which can itself absorb light for the purpose of providing the absorbed energy for catalyzing the polymerization reaction, is the η-cyclopentadienyl-tri(σ-hydrocarbyl)platinum compound according to formula (I), as disclosed in U.S. Pat. No. 8,088,878 B2, wherein this is linked to a hydrolyzable silyl group by an alkyl group, as indicated in claim 4 of this application. This catalyst can be excited by visible light in order to catalyze hydrosilylation reactions.

While in the case of other platinum-based catalysts there is a risk of the relatively small, non-polar molecules passing through the skin barrier, in the reaction with this catalyst an incorporation into the silicone matrix is effected without substantially altering the ligand structure or negatively impacting the catalyst activity. The vapour pressure of the platinum compound is also considerably lowered hereby, which considerably reduces the absorption via the respiratory system in the form of fine particles and a possible toxic effect associated therewith.

The cosmetic or pharmaceutical composition according to the invention contains at least one polymer precursor and a polymerization catalyst for the polymerization of the polymer precursor under the action of light.

The cosmetic or pharmaceutical composition of the invention preferably consists of a mixture of substances which contains both the polymer precursor and the polymerization catalyst, with the result that these two constituents are applied to the skin at the same time in the form of a ready-made mixture. In a specific embodiment, the cosmetic or pharmaceutical composition can, however, also consist of two or more separately available components, in which the polymer precursor and the polymerization catalyst are contained separately from one another. In this way the polymer precursor and the polymerization catalyst are either not mixed until shortly before application or are even applied successively to the skin, and thus are not brought into contact with one another and blended until in situ, i.e. immediately before application or even not until during application.

In particular embodiments the cosmetic or pharmaceutical composition can additionally contain emulsifiers, anti-ageing agents, a particle phase, an oil-water carrier phase, antioxidants, salts or combinations thereof.

The cosmetic or pharmaceutical composition according to the invention can be in the form of a gel, a cream, an ointment, a spray, a lotion, an oil-water emulsion, a hydrogel, a balm, a solution or a suspension.

In a particular embodiment of the invention, the cosmetic or pharmaceutical composition is in the form of an oil-in-water emulsion, wherein the total weight of the composition contains 5-70 wt.-% oil phase. The oil phase can contain one or more silicone oils which are selected from cyclic or linear dialkyl-, alkylaryl- and arylsiloxanes. Examples are cyclopentasiloxane, dimethylpolysiloxane, phenylphenylpolysiloxane and methylphenylpolysiloxane.

The oil phase can also contain one or more ester oils; ester oils of benzoic acid with linear or branched C₅₋₂₅ alkanols are particularly preferred.

Further cosmetic oils which can be contained in the oil phase are liquid paraffin oils, isoparaffin oils and synthetic hydrocarbons such as, for example, di-n-alkyl ether with a C₅₋₂₅ chain length.

Further oils according to the invention which can be contained in the cosmetic or pharmaceutical composition are linear or branched, saturated or unsaturated fatty alcohols with 6-30 carbon atoms or vegetable oils such as, for example, olive oil, soya oil, sesame oil, hazelnut oil, corn oil or jojoba oil.

The cosmetic or pharmaceutical composition according to the invention can furthermore contain 10-40 wt.-% of a dispersed particle phase, relative to its total weight. The particles of the particle phase have a number-average particle diameter of 1-100 μm, preferably 2-50 μm, particularly preferably 5-25 μm. An essential constituent of this dispersed particle phase is a silicone elastomer, which is present dispersed in the aqueous phase or in the emulsion and does not thicken the oil phase. Silicone elastomers of this type are described in more detail in EP 2 382 961 A2 and considerably improve the wrinkle concealing capacity of the composition according to the present invention.

Further specific embodiments of the invention are characterized in that, in addition to silicone particles, the particle phase contains further particles which are selected from the following particles: starch and starch derivatives, cellulose powders, polymer powders comprising polyolefins, polycarbonates, polyurethanes, polyamides, polyesters, polystyrenes, polyacrylates, polymethacrylates, (meth)acrylate or (meth)acrylate-vinylidene copolymers, Teflon or combinations of these.

In a further embodiment according to the invention, the composition contains 40-85 wt.-%, preferably 50-75 wt.-%, particularly preferably 51-60 wt.-% of aqueous phase, relative to the total weight of the composition according to the invention.

In addition to water, the aqueous phase can contain di- or polyols such as, for example, 1,2-propylene glycol, glycerol, butylene glycol, 1,6-hexanediol or polyethylene glycols as well as combinations of these. The aqueous phase can moreover contain inorganic salts such as, for example, sodium chloride and/or bases such as, for example, sodium hydroxide.

The composition according to the invention can contain at least one emulsifier, wherein in total 0.01 to 1 wt.-% emulsifier(s), preferably 0.1 to 0.5 wt.-%, particularly preferably 0.2 to 0.4 wt.-% are contained, relative to the total weight of the composition according to the invention.

Preferred emulsifiers are lecithins, silicone copolyols with ethylene oxide units, alkyl mono- and oligoglycosides with 8 to 22 carbon atoms in the alkyl radical and ethoxylated analogues thereof, ethoxylated sterols, partial esters of polyglycerols with 2 to 10 glycerol units and esterified with 1 to 4 saturated or unsaturated, linear or branched or cyclic, optionally hydroxylated C₈-C₃₀ fatty acid esters, as well as combinations of the named substances.

In an embodiment the cosmetic or pharmaceutical composition contains anti-ageing agents such as, for example, agents for stimulating collagen production, agents for stimulating glycosaminoglycan synthesis and proteoglycan synthesis, agents for stimulating integrin synthesis in the fibroblasts, agents for stimulating fructosamine-3-kinase and associated protein expression, agents for stimulating tensin expression, agents which promote the production of growth factors, agents which induce the proliferation of the fibroblasts, agents which induce the proliferation of keratinocytes, antioxidants, radical scavengers and combinations of the named compounds. Examples of such compounds are: plant extracts of Butea frondosa, Magnolia champaca, Vanilla planifolia, Cedrus atlantica, Canarium commune, Zingiber cassumunar Roxb., Chondrus crispus, Thermus thermophilus, Pisum sativum, Centella asiatica, Scenedesmus, Moringa pterygosperma, Hamamelis, Castanea sativa, Hibiscus sabdariffa, Polyanthes tuberosa, Argania spinosa, Aloe vera, Narcissus tazetta, or liquorice, vegetable oil of Citrus aurantium (neroli), α-hydroxy acids, in particular glycolic, lactic and cyclic acids, and esters thereof, β-hydroxy acids, in particular salicylic acid and derivatives thereof, hydrolysates of plant proteins, acylated oligopeptides, yeast extracts, in particular extracts of Saccharomyces cerevisiae, extracts of algae, in particular of brown algae, vitamins and derivatives thereof, in particular retinyl palmitates, ascorbic acid, ascorbyl glucosides, magnesium or sodium ascorbyl phosphate, ascorbyl palmitate, ascorbyl tetraisopalmitate, ascorbyl sorbate, tocopherol, tocopheryl acetate and tocopheryl sorbates, alkylated or phosphorylated esters of ascorbic acid, or of tocopherol or esters thereof, complexing agents, in particular EDTA salts, pH regulators, preservatives, in particular parabens and phenoxyethanol and combinations thereof.

According to the invention the polymerization of the material can be effected either in a purely light-induced and -driven process or by dual polymerization. By dual polymerization is meant the initiation of the polymerization process by light followed by chemical polymerization, or in reverse order. The mechanism of the chemical polymerization can be selected from a condensation reaction polymerization, an addition reaction polymerization, a ring-opening reaction polymerization, a catalytic polymerization and/or a radical polymerization.

According to the invention the composition can be used for wrinkle reduction, in that the composition applied to the treated area penetrates into the skin wrinkles, partially fills and, after the polymerization, fixes them in an advantageous position in order in this way to visually conceal the existing skin wrinkles.

According to the invention the composition can also generally be used for the tautening or fixation of the skin in a desired position. For this the skin is manually brought into a desired position and the composition is applied. Alternatively, first the composition is applied and then the skin is manually brought into a desired position. After polymerization of the composition the skin remains in the desired position. This may be desired, for example, in order to tauten the skin in the treated area for aesthetic reasons and, among other things, thus to reduce wrinkles intrinsically present in this area. However, it may also be desired to keep the skin in as taut and wrinkle-free a form as possible, e.g. overnight, in order to prevent the wrinkle formation due to unconscious facial expressions or a mechanical influence (e.g. pillow). In this way individual parts of the face can even be given a form other than the natural shape.

To enhance the above-named effect, the composition can additionally contain substances with an adhesive effect. Examples thereof are: self-adhesive polyurethanes, epoxy resins, natural resins, polyester resins, ABS resins, aminoplasts, phenolic resins, poly(methyl) methacrylates, polycyanoacrylates.

The composition according to the invention can also be used over a large area and in a greater layer thickness for body shaping, e.g. layers can be formed with the composition according to the invention which can take on the function of a bra.

The composition according to the invention can also be used over a large area and in a greater layer thickness in order to form a protective layer against environmental influences. For example with the composition according to the invention a continuous protective layer can be formed on the skin of the hands, which can take on the function of made-to-measure gloves.

According to the invention the composition can also be used for the application and fixing of make-up. For this the composition can contain additional decorative pigments or dyes. Examples thereof are metal-effect pigments, pearlescent pigments, luminescent pigments, natural organic pigments, synthetic organic pigments. Pigment- or dye-containing layers can also be used to cover unattractive scars or skin areas with pigmentary abnormality.

According to the invention the composition can also be used as a liquid plaster or ready-made highly elastic plaster. Due to the volume contraction during polymerization, a force is exerted on the wound edges and they are brought closer together. This ensures improved wound healing. Such plasters can be used as surgical wound closures, blister plasters, active-ingredient plasters or also as spray-on plasters.

According to the invention the composition can also be used for shaping and/or fixing body hairs and/or artificial hairs. Layers of the composition according to the invention applied directly to one's own, someone else's or artificial hair can be used for the shaping, and optionally also for colouring. In layers of the composition according to the invention applied to one's own skin or to one's own hairs, however, someone else's natural hair or artificial hairs can also be implanted in order to attach this hair to one's own skin or to one's own hairs.

Furthermore, the composition according to the invention can be used for the removal of hairs. For this the composition is applied to a skin area and following the polymerization the layer formed are peeled off. The above-named additives with adhesive properties can be utilized to enhance this effect.

According to the invention the composition can be used in combination with an active ingredient for producing a pharmaceutical preparation which is suitable for treating skin ageing and symptoms associated with skin ageing and for increasing the longevity of skin cells. Within the meaning of the present invention, a pharmaceutical composition can, for example, additionally contain a sirtuin activator, such as e.g. quercetin, piceatannol, resveratrol or isonicotinamide.

The formulations can contain all adjuvants and additives which are usually used in cosmetic or pharmaceutical preparations. In particular, those additives which have an effect on the physical properties of the active ingredients and/or vesicles and the stability thereof and/or serve to preserve the composition or active-ingredient composition come under the term “adjuvant” in the context of the present invention. Examples of such adjuvants are oils, alcohols, polyols, gel formers, buffers, preservatives, bactericides and germicides, complexing agents, thickeners or consistency agents.

Of course all components of the compositions, active-ingredient compositions and formulations according to the invention are pharmaceutically, cosmetically or dermatologically tolerated substances. Within the meaning of this invention, a substance is pharmaceutically, cosmetically or dermatologically tolerated if it is non-toxic and can be applied topically in most potential users without causing an undesired physiological reaction in the user, such as e.g. redness or the development of itching, spontaneously or after a while.

The composition according to the invention can either be used purely cosmetically (non-therapeutically), purely pharmaceutically (therapeutically) or as a combination of the two (cosmetically/pharmaceutically). Preferably it is used as a purely cosmetic composition for one of the above-named purposes, e.g. for visually concealing wrinkles and cracks and dryness of the skin.

In order to provide an elastic, wearable polymer layer on the skin for one of the above-named intended uses, it is proposed according to the invention to carry out the following process steps:
a) topical application of the polymer precursor to the skin area to be treated,
b) topical application of the polymerization catalyst to the skin area to be treated,
c) optionally topical application of a photosensitizer to the skin area to be treated, and
d) exposure of the skin area to be treated to light,
wherein the sequence in which the process steps are carried out can be varied, with
the proviso that process step d) is always the last process step.

A composition according to the invention in which the polymer precursor and the polymerization catalyst are present in a ready-made mixture is preferably used.

In these cases the process is simplified by the fact that only the following steps are to be carried out:
a) topical application of the mixture which contains the polymer precursor and the polymerization catalyst to the skin area to be treated, optionally accompanied by simultaneous or subsequent topical application of a photosensitizer to the skin area to be treated, and
b) exposure of the skin area to be treated to light.

Irrespective of which of the two processes is used, the exposure of the skin area to be treated to light is effected in the wavelength ranges named for the composition according to the invention for polymerization under the action of light.

For the purpose of original disclosure, it is pointed out that all features, as revealed to a person skilled in the art from the present description and the claims, even if they have been described specifically only in connection with particular further features, can be combined both individually and in any desired combinations with others of the features or groups of features disclosed here, unless this has been explicitly ruled out or chemical, physical-chemical, cosmetic, pharmacological or dermatological circumstances make such combinations impossible or meaningless. Merely for the sake of brevity and readability of the description, the comprehensive, explicit representation of all conceivable combinations of features is dispensed with here.

1. A cosmetic or pharmaceutical composition for topical application to the skin, comprising:
at least one polymer precursor; and
at least one polymerization catalyst, in the presence of which the at least one polymer precursor polymerizes under the action of light.

2. The cosmetic or pharmaceutical composition according to claim 1, wherein the at least one polymer precursor is a siloxane.

3. The cosmetic or pharmaceutical composition according to claim 1, wherein the at least one polymer precursor is a polyether-modified siloxane.

4. The cosmetic or pharmaceutical composition according to claim 1, wherein the at least one polymer precursor is selected from
a) a combination of at least one:
i) polyorganohydrosiloxane with the following structure: 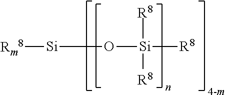
wherein R⁸ represent independent radicals selected from a group consisting of alkyl groups, cycloalkyl groups, phenyl groups, epoxide groups, isocyanate groups, amino groups, alcohol groups, ether groups, ester groups and hydrogen, provided that at least two, but not more than half of all R⁸ groups in the siloxane are hydrogens, and wherein m is 0, 1, 2 or 3 and n is a number with a value between 1 and 3000,
and at least one
ii) polyorganosiloxane with the following structure: 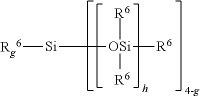
wherein R⁶ represent independent radicals selected from a group consisting of non-halogenated or halogenated ethylenically unsaturated groups, non-halogenated or halogenated alkyl groups, non-halogenated or halogenated cycloalkyl groups, epoxide groups, isocyanate groups, amino groups, alcohol groups, ether groups, ester groups, phenyl groups, provided that at least 70% of all R⁶ groups contain vinyl groups or other alkenyl groups and wherein h is a number with a value between 1 and 3000 and g is 0, 1, 2, or 3,
and
b) at least one polyorganohydrosiloxane with the following structure: 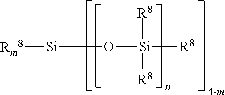
wherein R⁸ represent independent radicals selected from a group consisting of alkyl groups, cycloalkyl groups, phenyl groups, epoxide groups, isocyanate groups, amino groups, alcohol groups, ether groups, ester groups, hydrogen and non-halogenated or halogenated ethylenically unsaturated groups, provided that at least two, but not more than half of all R⁸ groups in the siloxane are hydrogens and furthermore provided that at least two of the R⁸ groups contain vinyl groups or alkenyl groups, wherein m is 0, 1, 2 or 3 and n is a number with a value between 1 and 3000.

5. The cosmetic or pharmaceutical composition according to claim 1, wherein the polymerization catalyst is a compound which comes under the general formula (I) 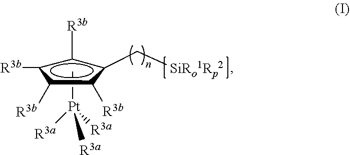
wherein
n=1−8,
o=0−2,
p=1−3 and o+p=3
and the substituents R¹ are identical or different and are in each case, independently of one another, a monovalent, unsubstituted or substituted, linear, cyclic or branched hydrocarbon radical, which contains aliphatically saturated and unsaturated or aromatic radicals and has 1-30 carbon atoms in which individual carbon atoms can be replaced by O, N, S or P atoms,
the substituents R² are hydrolyzable functional groups, identical or different from one another, selected from a group consisting of:
carboxy of the general formula —O—C(O)R⁴,
oxime of the general formula —O—N═CR⁴₂,
alkoxy of the general formula —OR⁴,
alkenyloxy of the general formula —O—R⁶,
amide of the general formula —NR⁴—C(O)R⁵,
amine of the general formula —NR⁴R⁵,
amineoxy of the general formula —O—NR⁴R⁵,
wherein
the radicals R⁴ are identical or different and are in each case, independently of one another, H, alkyl, aryl, arylalkyl or alkylaryl,
the substituents R⁵ are identical or different and are in each case, independently of one another, alkyl, aryl, arylalkyl, or alkylaryl,
R⁶ is a linear or branched, aliphatically unsaturated radical,
the substituents R³a are identical or different and are in each case, independently of one another, alkyl, aryl, arylalkyl or alkylaryl, wherein these have 1-30 carbon atoms, wherein the hydrogen atoms can be replaced by -Hal or —SiR₃³,
the substituents R³ are identical or different and are in each case, independently of one another, monovalent, unsubstituted or substituted, linear, cyclic or branched hydrocarbon radicals,
the substituents R^{3b} are identical or different and are in each case, independently of one another, hydrogen or a monovalent, unsubstituted or substituted, linear or branched hydrocarbon radical, wherein this contains an aliphatically saturated or unsaturated or aromatic radical and has 1-30 carbon atoms and in which the individual carbon atoms can be replaced by O, N, S or P atoms.

6. The cosmetic or pharmaceutical composition according to claim 1, wherein the polymerization catalyst is a compound which comes under the general formula (II), 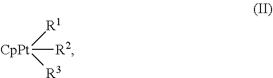
wherein
Cp represents a cyclopentadienyl group which is η-bonded to the platinum atom, wherein the cyclopentadienyl group is optionally mono- or polysubstituted by radicals which do not interfere with the hydrosilylation reaction, and
each of the radicals R¹, R² and R³ represents an aliphatic or aromatic group with 1-18 carbon atoms, wherein R¹, R² and R³ are a-bonded to the platinum atom,
and wherein the composition additionally contains at least one photosensitizer which is suitable for transferring the absorbed light energy to the polymerization catalyst.

7. The cosmetic or pharmaceutical composition according to claim 6, wherein the at least one photosensitizer is a compound which is selected from a group consisting of polycyclic aromatics with 2-5 rings, thioxanthones, 2-chlorothioxanthones, 2-isopropylthioxanthones, aromatic compounds with at least one ketone chromophore and combinations thereof.

8. The cosmetic or pharmaceutical composition according to claim 1, wherein the composition additionally contains a particle phase which consists of particles which are selected from a group consisting of: silicones, starch and starch derivatives, cellulose powders, polymer powders comprising polyolefins, polycarbonates, polyurethanes, polyamides, polyesters, polystyrenes, polyacrylates, polymethacrylates, (meth)acrylate or (meth)acrylate-vinylidene copolymers, Teflon or combinations thereof.

9. The cosmetic or pharmaceutical composition according to claim 1, wherein the composition contains decorative pigments and/or dyes selected from a group consisting of metal-effect pigments, pearlescent pigments, luminescent pigments, natural organic pigments or synthetic organic pigments.

10. A non-therapeutic method comprising applying, to skin, a cosmetic composition according to claim 1 for skin tautening, wrinkle reduction or for visually concealing skin wrinkles, cracks in the skin, dry skin and/or scar tissue of the skin and/or skin areas with pigmentary abnormality.

11. A non-therapeutic method comprising applying, to skin, a cosmetic composition according to claim 1 for shaping and/or fixing the skin in a desired position and/or for shaping and/or fixing one's own hair, someone else's hair or artificial hair.

12. A non-therapeutic method comprising applying, to skin, a cosmetic composition according to claim 1 for the removal of body hairs.

13. A non-therapeutic method comprising applying, to skin, a cosmetic composition according to claim 1, wherein the compositions is applied as a liquid plaster, ready-made elastic plaster, surgical wound closure, blister plaster, active-ingredient plaster or spray-on plaster.

14. A process for the production of an elastic, wearable polymer layer on the skin, wherein the process comprises the following process steps:
a) topical application of a polymer precursor, as defined in one of the preceding claims, to the skin area to be treated,
b) topical application of a polymerization catalyst, as defined in one of the preceding claims, to the skin area to be treated,
c) optionally topical application of a photosensitizer, as defined in one of the preceding claims, to the skin area to be treated, and
d) exposure of the skin area to be treated to light,
wherein the sequence in which the process steps are carried out can be varied, with the proviso that process step d) is always the last process step.

15. The process according to claim 14, wherein the exposure of the skin area to be treated to light according to process step d) is effected by irradiation with visible light of a wavelength in the range of 400-800 nm.
